(19) United States
(12) Patent Application Publication
(10) Pub. No.: US 2022/0308071 A1
(43) Pub. Date: Sep. 29, 2022

(54) METHODS AND COMPOSITIONS FOR DETERMINATION OF LIVER FIBROSIS

(71) Applicant: The Regents of the University of California, Oakland, CA (US)

(72) Inventors: Edward A. Dennis, La Jolla, CA (US); Rohit Loomba, San Diego, CA (US)

(21) Appl. No.: 17/618,257

(22) PCT Filed: Jun. 13, 2020
(86) PCT No.: PCT/US2020/037638

§ 371 Date: Dec. 10, 2021

Related U.S. Application Data
(60) Provisional application No. 62/861,263, filed on Jun. 13, 2019.

(51) Int. Cl. G01N 33/68; G01N 33/92; G01N 30/72

(52) U.S. Cl. CPC G01N 33/6893; G01N 33/92; G01N 30/7233; G01N 2800/085; G01N 2800/50

(57) ABSTRACT

The disclosure provides methods for determining liver fibrosis development, risk and prognosis.

### CROSS REFERENCE TO RELATED APPLICATIONS

This application claims priority of U.S. Provisional Application No. 62/861,263, filed Jun. 13, 2019, the disclosures of which are incorporated herein by reference.

### FIELD OF THE INVENTION

The invention relates in general to materials and methods to quantitate markers to determine liver disease and fibrosis.

### BACKGROUND

Fatty liver disease (or steatohepatitis) is often associated with excessive alcohol intake or obesity, but also has other causes such as metabolic deficiencies including insulin resistance and diabetes. Fatty liver results from triglyceride fat accumulation in vacuoles of the liver cells resulting in decreased liver function, and possibly leading to cirrhosis or hepatic cancer.

Non-alcoholic fatty liver disease (NAFLD) represents a spectrum of disease occurring in the absence of alcohol abuse.

There is a clinical need for a simple test to identify individuals with nonalcoholic fatty liver disease (NAFLD) in the population.

### SUMMARY

Eicosanoid and related docosanoid polyunsaturated fatty acids (PUFAs) and their oxygenated derivatives have been proposed as noninvasive lipidomic biomarkers of nonalcoholic steatohepatitis (NASH). The disclosure demonstrates the association between plasma eicosanoids and liver fibrosis and evaluates their utility in diagnosing and monitoring NASH-related fibrosis. The analysis used baseline eicosanoid data from 427 patients with biopsy confirmed NAFLD, and longitudinal measurements along with liver fibrosis staging from 63 patients with NASH and stage 2/3 fibrosis followed for 24 weeks in a phase 2 trial. At baseline, four eicosanoids were significantly associated with liver fibrosis stage: 11,12-DiHETE, tetranor 12-HETE, adrenic acid, and 14,15-DiHETE. Over 24-weeks of follow-up, a combination of changes in seven eicosanoids (5-HETE, 7,17-DHDPA, adrenic acid, arachidonic acid, EPA, 16-HDOHE, and 9-HODE) had good diagnostic performance for the prediction of 1 stage improvement in fibrosis (AUROC: 0.74; 95% CI: 0.62-0.87) and a combination of four eicosanoids (7,17-DHDPA, 14,15-DIHETRE, 9-HOTRE, and free adrenic acid) accurately predicted improvement in hepatic collagen content (AUROC: 0.72; 95% CI: 0.50-0.77). This disclosure provides markers and methods that plasma eicosanoids can serve as noninvasive biomarkers of liver fibrosis and can predict liver fibrosis improvement in NASH.

The disclosure provides a method of determining changes in liver fibrosis, comprising (a) obtaining a biological sample from the subject; (b) spiking the sample with deuterated standards; (c) extracting one or more eicosanoids selected from the group consisting of adrenic acid, 11,12-diHETE, tetranor 12-HETE, 14,15-diHETE and any combination thereof; (d) measuring the one or more eicosanoids using chromatography and/or gas chromatography mass spectroscopy; and (e) comparing the levels of adrenic acid, 11,12-diHETE, tetranor 12-HETE and/or 14,15-diHETE in the biological sample obtained from the subject to a control or prior sample, wherein a difference in the levels is indicative of a change in liver fibrosis. In one embodiment, the method comprises a diagnosticum comprising a set for control standard values, a set of deuterated internal standards and instructions for carrying out the method. In another embodiment, the biological sample is selected from the group consisting of blood, blood plasma and blood serum. In still another or further embodiment, the method further comprises measuring 5-HETE, 7,17-DHDPA, arachidonic acid, EPA, 16-HDOHE and 9-HODE. In still another or further embodiment, the method further comprises measuring additional eicosanoid in the sample obtained from the subject. In another or further embodiment of any of the foregoing, the one or more eicosanoids are measured by liquid chromatography. In still another or further embodiment of any of the foregoing, the one or more eicosanoids are measured by gas chromatography mass spectrometry. In yet another or further embodiment of any of the foregoing, the method further comprises determining the area under receiver operating characteristic curve (AUROC) based upon a ratio of the levels of the one or more eicosanoids matched with the deuterated standards of the same eicosanoid.

The disclosure also provides a method of determining liver fibrosis improvement, comprising (a) obtaining a biological sample from the subject; (b) spiking the sample with deuterated standards; (c) extracting one or more eicosanoids selected from the group consisting of 5-HETE, 7,17-DHDPA, adrenic acid, arachidonic acid, EPA, 16-HDOHE, 9-HODE and any combination thereof; (d) measuring the one or more eicosanoids using chromatography and/or gas chromatography mass spectroscopy; and (e) comparing the levels of 5-HETE, 7,17-DHDPA, adrenic acid, arachidonic acid, EPA, 16-HDOHE, and/or 9-HODE in the biological sample obtained from the subject to a control or prior sample, wherein a positive percent change in the levels is indicative of an improvement in liver fibrosis. In another embodiment, the biological sample is selected from the group consisting of blood, blood plasma and blood serum. In still another or further embodiment, the method further comprises measuring one or more of 11,12-diHETE, tetranor 12-HETE, 14,15-diHETE, 14-HDOHE, 9-HOTRE, DHA, and/or EPA. In still another or further embodiment, the one or more eicosanoids are measured by liquid chromatography. In yet another or further embodiment, the one or more eicosanoids are measured by gas chromatography mass spectrometry. In still another or further embodiment, the method further comprises determining the area under receiver operating characteristic curve (AUROC) based upon a ratio of the levels of the one or more eicosanoids matched with the deuterated standards of the same eicosanoid.

The disclosure also provides a method of determining an improvement in hepatic collagen content, comprising: (a) obtaining a biological sample from the subject; (b) spiking the sample with deuterated standards; (c) extracting one or more eicosanoids selected from the group consisting of 14-HDOHE; 7,17-DHDPA; 9HOTRE; adrenic acid; arachidonic acid; DHA; EPA; 14,15-DiHETRE and any combination thereof; (d) measuring the one or more eicosanoids using chromatography and/or gas chromatography mass spectroscopy; and (e) comparing the levels of 14-HDOHE; 7,17-DHDPA; 9HOTRE; adrenic acid; arachidonic acid; DHA; EPA; and/or 14,15-DiHETRE in the biological sample obtained from the subject to a control or prior sample, wherein a positive percent change in the levels is indicative of an improvement in liver fibrosis. In another embodiment, the biological sample is selected from the group consisting of blood, blood plasma and blood serum. In another or further embodiment, the method further comprises measuring one or more of 5-HETE, 16-HDOHE, and/or 9-HODE. In yet another or further embodiment, the one or more eicosanoids are measured by liquid chromatography. In yet another or further embodiment, the one or more eicosanoids are measured by gas chromatography mass spectrometry. In still another or further embodiment, the method further comprises determining the area under receiver operating characteristic curve (AUROC) based upon a ratio of the levels of the one or more eicosanoids matched with the deuterated standards of the same eicosanoid.

The disclosure also provides a method of determining improved prognosis of liver fibrosis comprising measuring eicosanoids selected from the group consisting of 5-HETE, 7,17-DHDPA, adrenic acid, arachidonic acid, EPA, 16-HDOHE, 9-HODE in a sample from a subject at a first time point; treating the subject with a therapeutic for the treatment of fibrotic liver disease; measuring eicosanoids selected from the group consisting of 5-HETE, 7,17-DHDPA, adrenic acid, arachidonic acid, EPA, 16-HDOHE, 9-HODE in a sample from a subject at a second time point after treating the subject; wherein if there is a positive percent increase in 5-HETE, 7,17-DHDPA, adrenic acid, arachidonic acid, EPA, 16-HDOHE, and/or 9-HODE, the therapeutic is treating liver fibrosis in the subject.

### BRIEF DESCRIPTION OF THE DRAWINGS

FIG. 1A-D shows baseline association of plasma eicosanoids with fibrosis stages. Plasma concentration of the 4 eicosanoids significantly associated with liver fibrosis stages A. 11,12-DIHETE, B. tetranor 12-HETE, C. adrenic acid D. 14,15-DIHETE are depicted as whisker plots across liver fibrosis stages. P-value were determined using the Jonckheere test.

FIG. 2A-B plasma eicosanoid changes are associated with changes in liver fibrosis and hepatic collagen. Median changes in plasma eicosanoids from baseline to week 24 of the most informative biomarkers stratified by A). Liver fibrosis changes: red bar participants with improvement of liver fibrosis ≥1 stage (n=20), green bar participants with no change in liver fibrosis (n=33), and blue bar participant with liver fibrosis worsening ≥1 stage (n=10). B). Hepatic collagen content (MQC) changes: red bar participants with improvement of MQC ≥20% (n=24), green bar participants with no change in MQC (n=14), and blue bar participant with MQC worsening ≥20% (n=25). P-value was determined using the Kruskal Wallis test.

### DETAILED DESCRIPTION

As used herein and in the appended claims, the singular forms “a,” “an,” and “the” include plural referents unless the context clearly dictates otherwise. Thus, for example, reference to “a derivative” includes a plurality of such derivatives and reference to “a subject” includes reference to one or more subjects and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

The publications discussed throughout the text are provided solely for their disclosure prior to the filing date of the present application. Nothing herein is to be construed as an admission that the inventors are not entitled to antedate such disclosure by virtue of prior disclosure.

“Biomarker” means a compound that is differentially present (i.e., increased or decreased) in a biological sample from a subject or a group of subjects having a first phenotype (e.g., having a disease or disease symptom) as compared to a biological sample from a subject or group of subjects having a second phenotype (e.g., not having the disease or disease symptom). A biomarker may be differentially present at any level, but is generally present at a level that is increased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more; or is generally present at a level that is decreased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent). A biomarker is preferably differentially present at a level that is statistically significant.

As used herein, “biomarker level” and “level” refer to a measurement that is made using any analytical method for detecting the biomarker in a biological sample and that indicates the presence, absence, absolute amount or concentration, relative amount or concentration, titer, a level, an expression level, a ratio of measured levels, or the like, of, for, or corresponding to the biomarker in the biological sample. The exact nature of the “level” depends on the specific design and components of the particular analytical method employed to detect the biomarker.

As used herein, “detecting” or “determining” with respect to a biomarker level includes the use of both the instrument used to observe and record a signal corresponding to a biomarker level and the material(s) required to generate that signal. In various embodiments, the level is detected using any suitable method, including fluorescence, chemiluminescence, surface plasmon resonance, surface acoustic waves, mass spectrometry, infrared spectroscopy, Raman spectroscopy, atomic force microscopy, scanning tunneling microscopy, electrochemical detection methods, nuclear magnetic resonance, quantum dots, and the like.

“Diagnose”, “diagnosing”, “diagnosis”, and variations thereof refer to the detection, determination, or recognition of a health status or condition of an individual on the basis of one or more signs, symptoms, data, or other information pertaining to that individual. The health status of an individual can be diagnosed as healthy/normal (i.e., a diagnosis of the absence of a disease or condition) or diagnosed as ill/abnormal (i.e., a diagnosis of the presence, or an assessment of the characteristics, of a disease or condition). The terms “diagnose”, “diagnosing”, “diagnosis”, etc., encompass, with respect to a particular disease or condition, the initial detection of the disease; the characterization or classification of the disease; the detection of the progression, remission, or recurrence of the disease; and the detection of disease response after the administration of a treatment or therapy to the individual. The diagnosis of fibrosis includes distinguishing individuals who have fibrosis from individuals who do not. The diagnosis of liver fibrosis includes distinguishing individuals who have liver fibrosis from individuals who do not have liver fibrosis.

A “reference level” or “reference sample level” of a biomarker means a level of the biomarker that is indicative of a particular disease state, phenotype, or predisposition to developing a particular disease state or phenotype, or lack thereof, as well as combinations of disease states, phenotypes, or predisposition to developing a particular disease state or phenotype, or lack thereof. A “positive” reference level of a biomarker means a level that is indicative of a particular disease state or phenotype. A “negative” reference level of a biomarker means a level that is indicative of a lack of a particular disease state or phenotype. A “reference level” of a biomarker may be an absolute or relative amount or concentration of the biomarker, a presence or absence of the biomarker, a range of amount or concentration of the biomarker, a minimum and/or maximum amount or concentration of the biomarker, a mean amount or concentration of the biomarker, and/or a median amount or concentration of the biomarker; and, in addition, “reference levels” of combinations of biomarkers may also be ratios of absolute or relative amounts or concentrations of two or more biomarkers with respect to each other. Appropriate positive and negative reference levels of biomarkers for a particular disease state, phenotype, or lack thereof may be determined by measuring levels of desired biomarkers in one or more appropriate subjects, and such reference levels may be tailored to specific populations of subjects (e.g., a reference level may be age-matched or gender-matched so that comparisons may be made between biomarker levels in samples from subjects of a certain age or gender and reference levels for a particular disease state, phenotype, or lack thereof in a certain age or gender group). Such reference levels may also be tailored to specific techniques that are used to measure levels of biomarkers in biological samples (e.g., LC-MS, GC-MS, etc.), where the levels of biomarkers may differ based on the specific technique that is used. A “control level” of a target molecule refers to the level of the target molecule in the same sample type from an individual that does not have the disease or condition, or from an individual that is not suspected of having the disease or condition. A “control level” of a target molecule need not be determined each time the present methods are carried out, and may be a previously determined level that is used as a reference or threshold to determine whether the level in a particular sample is higher or lower than a normal level. In some embodiments, a control level in a method described herein is the level that has been observed in one or more subjects (i.e., a population) without fibrosis. In some embodiments, a control level in a method described herein is the level that has been observed in one or more subjects with fibrosis. In some embodiments, a control level in a method described herein is the average or mean level, optionally plus or minus a statistical variation that has been observed in a plurality of normal subjects, or subjects with fibrosis or without fibrosis. In some instances, a level of a biomarker may be positively increased relative to a control, wherein the increase is indicative of an improvement.

Eicosanoids, docosanoid polyunsaturated fatty acids (PUFAs) and related metabolites, sometimes referred to as oxylipins, are a group of structurally diverse metabolites that are shown herein to be lipidomic biomarkers for non-alcoholic steatohepatitis (NASH). They are locally acting bioactive signaling lipids that regulate a diverse set of homeostatic and inflammatory processes.

Bioactive lipids include a number of molecules whose concentrations or presence affect cellular function. Bioactive lipids, as used herein, include phospholipids, sphingolipids, lysophospholipids, ceramides, diacylglycerol, eicosanoids, steroid hormones and the like. Eicosanoids and related metabolites, sometimes referred to as oxylipins, are a group of structurally diverse metabolites that derive from the oxidation of polyunsaturated acids (PUFAs) including arachidonic acid (AA), linoleic acid, alpha and gamma linolenic acid, dihomo gamma linolenic acid, eicosapentaenoic acid and docosahexaenoic acid. They are locally acting bioactive signaling lipids that regulate a diverse set of homeostatic and inflammatory processes. Given the important regulatory functions in numerous physiological and pathophysiological states, the accurate measurement of eicosanoids and other oxylipins is of great clinical interest and lipidomics is now widely used to screen effectively for potential disease biomarkers.

The biosynthesis of eicosanoids and oxylipins involves the action of multiple enzymes organized into a complex and intertwined lipid-anabolic network. Generally, the enzymatic formation of eicosanoids requires free fatty acids as substrates; thus, the pathway is initiated by the hydrolysis of phospholipids (PLs) by phospholipase A₂ upon physiological stimuli. The hydrolyzed PUFAs are then processed by three enzyme systems: cyclooxygenases (COX), lipoxygenases (LOX), and cytochrome P450 enzymes (CYP450). Each of these enzyme systems produces unique collections of oxygenated metabolites that function as end-products or as intermediates for a cascade of downstream enzymes. The resulting eicosanoids exhibit diverse biological activities, half-lives and utilities in regulating many physiological processes in health and disease including the immune response, inflammation, and homeostasis. Additionally, non-enzymatic processes can produce oxidized PUFA metabolites via free radical reactions giving rise to isoprostanes and other oxidized fatty acids.

Eicosanoids act locally in an autocrine or paracrine fashion and signal by binding to G-protein-coupled receptors or act intracellularly via various peroxisome proliferator-activating receptors. For optimal biological activity, these mediators need to be present in their free, non-esterified form. However, a number of studies reported that a portion of eicosanoids are naturally esterified and can also be contained in cell membrane lipids, including PLs, in the form of esters. The role of esterified eicosanoids is not clear but they may be signaling molecules in their own right or serve as a cellular reservoir for the rapid release upon cell stimulation.

Two potential mechanisms for the formation of eicosanoids-containing PLs have been proposed: (i) direct oxidation of PUFAs on the intact PLs, and (ii) re-acylation of preformed free oxylipins into lysoPLs. Cyclooxygenases require free fatty acid as substrate and show little activity toward PUFAs in intact PLs. A number of subsequent studies support the concept that prostaglandins are first formed enzymatically and then incorporated into PLs by the sequential actions of long-chain acyl-CoA synthases and lysophospholipid acyltransferases. Additionally, preformed fatty acid epoxides, including the regioisomers of epoxyeicosatrienoic acid (EET), are effectively incorporated primarily into the phospholipid fraction of cellular lipids, presumably via CoA-dependent mechanisms.

Non-alcoholic fatty liver disease (NAFL or NAFLD) represents a spectrum of disease occurring in the absence of alcohol abuse. NAFLD is characterized by the presence of steatosis (fat in the liver) and may represent a hepatic manifestation of the metabolic syndrome (including obesity, diabetes and hypertriglyceridemia). NAFLD is linked to insulin resistance, it causes liver disease in adults and children and may ultimately lead to cirrhosis (Skelly et al., J Hepatol., 35: 195-9, 2001; Chitturi et al., Hepatology, 35(2):373-9, 2002). The severity of NAFLD ranges from the relatively benign isolated predominantly macrovesicular steatosis (i.e., nonalcoholic fatty liver or NAFL) to non-alcoholic steatohepatitis (NASH) (Angulo et al., J Gastroenterol Hepatol, 17 Suppl:S186-90, 2002). NASH is characterized by the histologic presence of steatosis, cytological ballooning, scattered inflammation and pericellular fibrosis (Contos et al., Adv Anat Pathol., 9:37-51, 2002). Hepatic fibrosis resulting from NASH may progress to cirrhosis of the liver or liver failure, and in some instances may lead to hepatocellular carcinoma.

The degree of insulin resistance (and hyperinsulinemia) correlates with the severity of NAFLD, being more pronounced in patients with NASH than with simple fatty liver (Sanyal et al., Gastroenterology, 120(5):1183-92, 2001). As a result, insulin-mediated suppression of lipolysis occurs and levels of circulating fatty acids increase. Two factors associated with NASH include insulin resistance and increased delivery of free fatty acids to the liver. Insulin blocks mitochondrial fatty acid oxidation. The increased generation of free fatty acids for hepatic re-esterification and oxidation results in accumulation of intrahepatic fat and increases the liver's vulnerability to secondary insults.

The prevalence of NAFLD in children is unknown because of the requirement of histologic analysis of liver in order to confirm the diagnosis (Schwimmer et al., Pediatrics, 118(4):1388-93, 2006). However, estimates of prevalence can be inferred from pediatric obesity data using hepatic ultra-sonongraphy and elevated serum transaminase levels and the knowledge that 85% of children with NAFLD are obese. Data from the National Health and Nutrition Examination Survey has revealed a threefold rise in the prevalence of childhood and adolescent obesity over the past 35 years; data from 2000 suggests that 14-16% children between 6-19 yrs age are obese with a BMI >95% (Fishbein et al., J Pediatr. Gastroenterol. Nutr., 36(1):54-61, 2003), and also that fact that 85% of children with NAFLD are obese.

In patients with histologically proven NAFLD, serum hepatic aminotransferases, specifically alanine aminotransferase (ALT), levels are elevated from the upper limit of normal to 10 times this level (Schwimmer et al., J Pediatr., 143(4):500-5, 2003; Rashid et al., J Pediatr Gastroenterol Nutr., 30(1):48-53, 2000). The ratio of ALT/AST (aspartate aminotransferase) is >1 (range 1.5-1.7) which differs from alcoholic steatohepatitis where the ratio is generally <1. Other abnormal serologic tests that may be abnormally elevated in NASH include gamma-glutamyltransferase (gamma-GT) and fasting levels of plasma insulin, cholesterol and triglyceride.

The exact mechanism by which NAFLD develops into NASH remains unclear. Because insulin resistance is associated with both NAFLD and NASH, it is postulated that other additional factors are also required for NASH to arise. This is referred to as the “two-hit” hypothesis (Day C P. Best Pract. Res. Clin. Gastroenterol., 16(5):663-78, 2002) and involves, firstly, an accumulation of fat within the liver and, secondly, the presence of large amounts of free radicals with increased oxidative stress. Macrovesicular steatosis represents hepatic accumulation of triglycerides, and this in turn is due to an imbalance between the delivery and utilization of free fatty acids to the liver. During periods of increased calorie intake, triglyceride will accumulate and act as a reserve energy source. When dietary calories are insufficient, stored triglycerides (in adipose) undergo lipolysis and fatty acids are released into the circulation and are taken up by the liver. Oxidation of fatty acids will yield energy for utilization.

The eicosanoid biosynthetic pathway includes over 100 bioactive lipids and relevant enzymes organized into a complex and intertwined lipid-signaling network. Biosynthesis of polyunsaturated fatty acid (PUFA) derived lipid mediators is initiated via the hydrolysis of phospholipids by phospholipase A₂ (PLA₂) upon physiological stimuli. These PUFA including arachidonic acid (AA), dihomo-gamma-linolenic acid (DGLA), eicosapentaenoic acid (EPA), and docosahexaenoic acid (DHA) are then processed by three enzyme systems: lipoxygenases (LOX), cyclooxygenases (COX) and cytochrome P450s, producing three distinct lineages of oxidized lipid classes. These enzymes are all capable of converting free arachidonic acid and related PUFA to their specific metabolites and exhibit diverse potencies, half-lives and utilities in regulating inflammation and signaling. Additionally, non-enzymatic processes can result in oxidized PUFA metabolites including metabolites from the essential fatty acids linoleic (LA) and alpha-linolenic acid (ALA).

Eicosanoids, which are key regulatory molecules in metabolic syndromes and the progression of hepatic steatosis to steatohepatitis in nonalcoholic fatty liver disease (NAFLD), act either as anti-inflammatory agents or as pro-inflammatory agents. Convincing evidence for a causal role of lipid peroxidation in steatohepatitis has not been unequivocally established; however, a decade of research has strongly suggested that these processes occur and that oxidative-stress is associated with hepatic toxicity and injury. As discussed above, nonalcoholic fatty liver disease (NAFLD) encompasses a wide spectrum of histological cases associated with hepatic fat over-accumulation that range from nonalcoholic fatty liver (NAFL) to nonalcoholic steatohepatitis (NASH). It is distinguished from NAFL by evidence of cytological ballooning, inflammation, and higher degrees of scarring and fibrosis. Hence, NASH is a serious condition, and approximately 10-25% of inflicted patients eventually develop advanced liver disease, cirrhosis, and hepatocellular carcinoma.

Thus, it is important to differentiate NASH from NAFL as well as NASH associated liver fibrosis. At the present time, the gold standard technique for the diagnosis of NASH or determining the extent of fibrosis is a liver biopsy examination, which is recognized as the only reliable method to evaluate the presence and extent of necro-inflammatory changes, presence of ballooning and fibrosis in liver. However, liver biopsy is an invasive procedure with possible serious complications and limitations. Reliable noninvasive methods are therefore needed to avoid the sampling risks. It is proposed that differences in plasma levels of free eicosanoids can distinguish NAFL from NASH based on studies of well-characterized patients with biopsy substantiated NAFL and NASH.

Cyclooxygenase-2 (COX-2), a key enzyme in eicosanoid metabolism, is abundantly expressed in NASH, which promotes hepatocellular apoptosis in rats. Others have reported that oxidized lipid products of LA including 9-hydroxyoctadienoic acid (9-HODE), 13-HODE, 9-oxooctadienoic acid (9-oxoODE), and 13-oxoODE as well as of arachidonic acid 5-hydroxyeicosa-tetraenoic acid (5-HETE), 8-HETE, 11-HETE, and 15-HETE are linked to histological severity in nonalcoholic fatty liver disease.

Free fatty acids are cytotoxic; thus the majority of all fatty acids in mammalian systems are esterified to phospholipids and glycerolipids as well as other complex lipids. Similarly, oxygenated metabolites of fatty acids can exist either in their free form or esterified to complex lipids. To capture all esterified eicosanoids for analysis, a saponification step is used to release them.

The disclosure provides methods, kits and compositions useful for determining the development and monitoring of liver fibrosis. Such methods will help in identifying or monitoring liver fibrosis and treatment of disease. Moreover, the methods reduce biopsy risks associated with liver biopsies currently used in diagnosis. The amount of a specific biomarker can be compared to normal standard sample levels (i.e., those lacking any liver disease) or can be compared to levels obtained from a diseased population (e.g., populations with clinically diagnosed NASH or NAFL).

As described below and elsewhere herein LC-MS/MS protocols are described to demonstrate that plasma or serum levels of oxylipins can be used as biomarkers to identify subjects having or at risk of having liver fibrosis. In this method, a panel of oxylipins that, when used together, can discriminate controls from fibrosis as well as provide prognosis of fibrosis of the liver with a high degree of certainty.

The disclosure includes the measurements of bioactive lipids. In some embodiments, methods were used to measure the “free” oxylipins present in plasma or serum, not those appearing after alkaline hydrolysis. In other embodiment, the sum total of esterified and free oxylipins are used by treating the sample with alkali (e.g., KOH) at mild concentrations such that the oxylipins are not degraded (e.g., 0.1M-0.6M KOH). Thus, the methods and compositions comprise modified eicosanoids and PUFAs in the diagnosis. As such, the biomarkers are manipulated from their natural state by chemical modifications to provide a derived biomarker that is measured and quantitated.

For example, eicosanoids and specifically PGs are sensitive to alkaline-induced degradation. Thus, experiments presented herein were performed to minimize degradation of lipid metabolites during alkaline treatment and to identify specific eicosanoids and related oxidized PUFAs that are released intact from esterified lipids and which can be quantitatively measured.

Levels of free eicosanoids and PUFA metabolites can be expressed as AUROC (Area under Receiver Operating Characteristic Curve). AUROC is determined by measuring levels of free eicosanoids and PUFA metabolites by stable isotope dilution. Briefly, identical amounts of deuterated internal standards are added to each sample and to all the primary standards used to generate standard curves. Levels of eicosanoids and PUFA metabolites are calculated by determining the ratios between endogenous metabolite and matching deuterated internal standards. Ratios are converted to absolute amounts by linear regression. Individual eicosanoid metabolites are assessed for diagnostic test performances and capability to differentiating between NAFL and NASH using statistical analyses including chi-square test, t-test and AUROC.

The method of the disclosure comprises determining the level of one or more free eicosanoids and/or polyunsaturated fatty acid (PUFA) metabolites in a sample of a patient. As used herein, the term “sample” refers to any biological sample from a patient. Examples include, but are not limited to, saliva, hair, skin, tissue, sputum, blood, plasma, serum, vitreal, cerebrospinal fluid, urine, sperm and cells. In one embodiment, the sample is a plasma sample. In another embodiment, the sample is a serum sample.

Lipids are extracted from the sample, as detailed further in the Examples. The identity and quantity of eicosanoids and/or PUFA metabolites in the extracted lipids is first determined and then compared to suitable controls. The determination may be made by any suitable lipid assay technique, such as a high throughput including, but not limited to, spectrophotometric analysis (e.g., colorimetric sulfo-phospho-vanillin (SPV) assessment method of Cheng et al., Lipids, 46(1):95-103 (2011)). Other analytical methods suitable for detection and quantification of lipid content will be known to those in the art including, without limitation, ELISA, NMR, UV-Vis or gas-liquid chromatography, HPLC, UPLC and/or MS or RIA methods enzymatic based chromogenic methods. Lipid extraction may also be performed by various methods known to the art, including the conventional method for liquid samples described in Bligh and Dyer, Can. J. Biochem. Physiol., 37, 91 1 (1959).

In one embodiment, serum is obtained from a subject suspected of having or having non-alcoholic fatty liver disease. The serum may be stored at −80° C. or used immediately for analysis. The serum is spiked with suitable deuterated standards. In one embodiment, 26 deuterated standards are spiked into the sample. For eicosanoid extraction, the sample is then brought to the desired volume with methanol and purified by solid phase extraction and the eicosanoids eluted with 100% methanol. The eluent is dried under vacuum, dissolved in buffer comprising water-acetonitrile and acetic acid (60:40:0.02 v/v/v).

In one embodiment, free fatty acids were analyzed by spiking serum from the subject with deuterated fatty acid standards and then isolating the free fatty acids by selective extraction with methanol and isooctane. The extracted fatty acids are then derivatized and analyzed by gas chromatography and MS.

In another embodiment, eicosanoids in the plasma are analyzed by quantified LC/MS/MS. The eicosanoids are separated by reverse phase chromatography. The eluted samples are integrated into a mass spectrometer. The eicosanoids were measured using electrospray ionization in negative ion mode and multiple reaction monitoring (MRM). The eicosanoids are identified by matching their MRM signal and chromatographic retention time with those of pure identical standards.

The disclosure describes 4 plasma eicosanoids significantly associated with fibrosis stage. Furthermore, using data from a multicenter phase 2 trial of selonsertib in patients with NASH and stage 2 or 3 fibrosis, the disclosure shows that changes in individual plasma eicosanoids were associated with improvement in both histological liver fibrosis stage and hepatic collagen content. The combination of these individual eicosanoids provides a diagnostic performance for the prediction of liver fibrosis improvement and a diagnostic for the prediction of hepatic collagen improvement. This demonstrates that plasma eicosanoids can serve as noninvasive biomarkers of liver fibrosis and improvement of liver fibrosis in patients with NAFLD.

For example, over 24-weeks of follow-up, a combination of changes in seven eicosanoids (5-HETE, 7,17-DHDPA, adrenic acid, arachidonic acid, EPA, 16-HDOHE, and 9-HODE) had good diagnostic performance for the prediction of ≥1 stage improvement in fibrosis (AUROC: 0.74; 95% CI: 0.62-0.87) and a combination of four eicosanoids (7,17-DHDPA, 14,15-DiHETRE, 9-HOTRE, and free adrenic acid) accurately predicted improvement in hepatic collagen content (AUROC: 0.72; 95% CI: 0.50-0.77). This demonstrates that plasma eicosanoids can serve as noninvasive biomarkers of liver fibrosis and can predict liver fibrosis improvement in NASH.

Several studies have previously reported alterations of plasma eicosanoids associated with the presence of NASH that are less likely to be found in patients with NAFL or in healthy controls. The present disclosure assesses associations between plasma eicosanoids and liver fibrosis in NAFLD. The disclosure demonstrates that certain eicosanoids are present in a common pathway leading to both NASH and liver fibrosis in patients with NAFLD.

This disclosure for the first time demonstrates an association between longitudinal changes in plasma eicosanoids and changes in histological stage of fibrosis or hepatic collagen content. The association between eicosanoid levels and fibrosis does not necessarily mean that eicosanoids have a causal role in the development of NASH and liver fibrosis; changes in eicosanoid levels may in some cases simply reflect alterations in pathways associated with disease activity. However, adrenic acid, arachidonic acid (AA), and DHA, which were identified among the most informative biomarkers to predict improvement of both liver fibrosis and hepatic collagen content, have been shown to be involved in the pathogenesis of NAFLD. AA is the precursor of eicosanoids through 3 main pathways including cytochrome P450s, cyclooxygenases, and lipoxygenases. Recent reports show that an increased release of AA from the phospholipid membrane and production of eicosanoid species in the liver of NASH promote inflammation and cell injury. In addition, an increase in plasma adrenic acid levels has been reported in children with hepatic steatosis and adrenic acid accumulation contributes to disease progression in NAFLD in mice. Finally, alterations in plasma DHA concentrations have been reported in patients with NASH and studies suggest that dietary supplementation of DHA may be effective at lowering liver fat content in NAFLD. Hence, changes in combination ‘panels’ of these eicosanoids could be a more direct measure of disease activity and may be better biomarkers of histological improvement in NAFLD patients than classic indirect markers of liver fibrosis such as alanine aminotransferase, aspartate aminotransferase, and platelet count.

There are several notable strengths of the data including the use of well-characterized cohorts from clinical trials with paired liver biopsies centrally read by a single, experienced hepatopathologist. In addition, the large sample size of the cohort for the baseline assessment (n=427) including all stages of liver fibrosis enabled the study of associations between plasma eicosanoids and stages of fibrosis. Further, the quantitative assessment using deuterated standards and LC/MS/MS provides a comprehensive profiling of plasma eicosanoids and free fatty acids.

The disclosure, using well-characterized cohorts, demonstrates that plasma eicosanoids are associated with liver fibrosis in NAFLD and that changes in plasma eicosanoids can predict liver fibrosis improvement.

It is to be understood that while the disclosure has been described in conjunction with specific embodiments thereof, that the foregoing description as well as the examples which follow are intended to illustrate and not limit the scope of the disclosure. Other aspects, advantages and modifications within the scope of the disclosure will be apparent to those skilled in the art to which the disclosure.

### EXAMPLES

### Example 1

Baseline assessment: A cross-sectional analysis of baseline data from 427 participants was performed for baseline plasma eicosanoid assessment and liver biopsy. Briefly, adult patients 18 to 65 years of age with body mass index (BMI) of at least 18 kg/m² were eligible for enrollment and were screened from November 2012 to October 2016. Patients were required to have chronic liver disease from NASH (defined histologically as macrovesicular steatosis involving >5% of hepatocytes with associated lobular inflammation) and fibrosis stage 3 or 4 (bridging fibrosis) based on a modified Ishak classification. All patients had to have aspartate and alanine aminotransferase values no higher than 10 times the upper limit of normal and serum creatinine level lower than 2.0 mg/dL based on central laboratory values.

Patients with any of the following conditions were excluded: any history of hepatic decompensation, including ascites, hepatic encephalopathy, or variceal hemorrhage; weight-reduction surgery in the prior 5 years; infection with the hepatitis B or C virus; alcohol consumption greater than 21 ounces/week for men or greater than 14 ounces/week for women; positive screen result for illegal drug use; or clinically significant cardiac disease. In addition, patients were excluded if they had a Child-Pugh-Turcotte score higher than 7, a Model for End-Stage Liver Disease score higher than 12, or a history of solid organ transplantation.

Longitudinal assessment: Longitudinal data—plasma eicosanoid, liver biopsy and hepatic collagen content were assessed at baseline and week 24 from 63 NASH patients with stage 2 or 3 fibrosis enrolled in a phase 2 study of selonsertib. Briefly, adult patients 18 to 70 years of age were enrolled at 23 sites in the United States and Canada from Jun. 8, 2015 to Mar. 31, 2016. To be eligible, patients were required to have a liver biopsy within 3 months of screening consistent with a diagnosis of NASH and stage 2 or stage 3 fibrosis according to the NASH Clinical Research Network (CRN) Histologic Scoring System. All patients had a NAFLD Activity Score (NAS) of 5 or higher, with a score of at least 1 point for each of the three components (steatosis, hepatocellular ballooning, and lobular inflammation). All patients had at least 3 of the following features of the metabolic syndrome: abdominal obesity, hypertension, elevated fasting glucose, elevated levels of serum triglycerides, or low levels of high-density lipoprotein cholesterol. Patients were randomly assigned in a 2:2:1:1:1 ratio to receive 24 weeks of treatment with 6 mg or 18 mg of selonsertib, 6 mg or 18 mg of selonsertib with 125 mg of simtuzumab, or 125 mg of simtuzumab alone. Selonsertib was administered orally once daily and simtuzumab was administered as weekly subcutaneous injections. After 24 weeks of treatment, 32% of the participants included in this study had at least a 1-stage reduction in fibrosis, 52% had no change in fibrosis stage and 16% had an increase of at least 1-stage in fibrosis. In addition, 38% of the participants had at least 20% reduction in hepatic collagen content, 22% no significant change in hepatic collagen content and 40% had an increase of at least 20% in hepatic collagen content. Treatment groups were combined for this analysis.

Histology: Biopsy samples of all participants were centrally read by a single experienced pathologist who was blinded to treatment assignment, but not to biopsy sequence. Histologic assessments included the adequacy of the biopsy specimen, confirmation of the diagnosis, fibrosis staged according to a modified Ishak classification and the NASH CRN system. Biopsy specimens were graded according to the NAFLD Activity Score (NAS), a standardized grading system for steatosis (on a scale of 0-3), lobular inflammation (on a scale of 0-3), and hepatocellular ballooning (on a scale of 0-2), with higher scores indicating increasing disease activity. Computer-assisted morphometry was also used to quantify hepatic collagen and fat content using picrosirius red-stained liver sections, as well as a smooth muscle actin (a-SMA) expression.

Baseline assessment: the primary outcome was the individual stage of fibrosis; the secondary outcome was the presence of advanced fibrosis (stage 3 or 4 according to the NASH CRN system).

Longitudinal assessment: the primary outcome was improvement in liver fibrosis, defined as a ≥1-stage reduction of fibrosis. The secondary outcome was improvement in hepatic collagen content as defined by a relative reduction of at least 20% from baseline to Week 24.

Lipid extraction: Serum samples for lipidomic profiling were obtained within 90 days of the liver biopsy. All serum samples were stored at −80° C., thawed once, and immediately used for free fatty acid and eicosanoid isolation. Briefly, 50 μl plasma was spiked with a cocktail of 26 deuterated internal standards (individually purchased from Cayman Chemicals, Ann Arbor, Mich.) and brought to a volume of 1 ml with 10% methanol. The samples were then purified by solid phase extraction on Strata-X columns (Phenomenex, Torrance, Calif.), using an activation procedure consisting of consecutive washes with 3 ml of 100% methanol followed by 3 ml of water. The eicosanoids were then eluted with 1 ml of 100% methanol, and the eluent was dried under vacuum, dissolved in 50 μl of buffer A (consisting of water-acetonitrile-acetic acid, 60:40:0.02 [v/v/v]), and immediately used for analysis as follows: For free fatty acids analysis, 50 μl of serum was spiked with deuterated fatty acid standards, and the free fatty acids were isolated by selective extraction with methanol and isooctane. The extracted fatty acids were derivatized and analyzed by gas chromatography and MS.

Reverse phase LC/MS: Eicosanoids in plasma were analyzed and quantified by LC/MS/MS. Briefly, eicosanoids were separated by reverse-phase chromatography using a 1.7 μM 2.1×100 mm BEH Shield Column (Waters, Milford, Mass.) and an Acquity UPLC system (Waters). The column was equilibrated with buffer A, and 5 μl of sample was injected via the autosampler. Samples were eluted with a step gradient starting with 100% buffer A for 1 min, then to 50% buffer B (consisting of 50% acetonitrile, 50% isopropanol, and 0.02% acetic acid) over a period of 3 min, and then to 100% buffer B over a period of 1 min. The LC was interfaced with an IonDrive Turbo V ion source, and mass spectral analysis was performed on a triple quadrupole AB SCIEX 6500 QTrap mass spectrometer (AB SCIEX, Framingham, Mass.). Eicosanoids were measured using electrospray ionization in negative ion mode and multiple reaction monitoring (MRM) using the most abundant and specific precursor ion/product ion transitions to build an acquisition method capable of detecting 158 analytes and 26 internal standards. The ionspray voltage was set at −4,500 V at a temperature of 550° C. Collisional activation of the eicosanoid precursor ions was achieved with nitrogen as the collision gas with the declustering potential, entrance potential, and collision energy optimized for each metabolite. Eicosanoids were identified by matching their MRM signal and chromatographic retention time with those of pure identical standards.

Quantitation of lipids: Eicosanoids were quantitated by the stable isotope dilution method. Briefly, identical amounts of deuterated internal standards were added to each sample and to all the primary standards used to generate standard curves. To calculate the amount of eicosanoids and free fatty acids in a sample, ratios of peak areas between endogenous metabolite and matching deuterated internal standards were calculated. Ratios were converted to absolute amounts by linear regression analysis of standard curves generated under identical conditions.

Data preparation: Out of the 161 eicosanoids measured in the LipoNexus platform, 34 were selected for further analyses based on: 1) 75% samples were ≥LLOQ (31 metabolites), and 2) previous evidence for relevance in NASH based upon the pilot study (3 metabolites).

Baseline analysis of the association of individual plasma eicosanoids with fibrosis stage was assessed using Jonckheere-Terpstra (ordinal stage categories) trend tests. The longitudinal analysis of the association of individual markers with binary response (e.g. improvement in fibrosis stage) was assessed using Wilcoxon tests. The performance of individual markers for the detection of liver fibrosis or improvement in fibrosis or hepatic collagen content was assessed using area under the receiver operating characteristics curve (AUROC) based on the whole dataset and 5-fold cross-validation repeated 100 times. An ad hoc approach was performed to combine markers for monitoring improvement responses. Specifically, plasma eicosanoids were considered if their AUROC (based on the full dataset) or Wilcoxon p-value for change from baseline to Week 24 from univariate analyses passed certain thresholds (e.g. AUROC ≥0.65 or Wilcoxon ≤0.1, and ≥75% of samples were above limit of quantitation [ALOQ]). Additionally, a systematic two-step approach was considered for monitoring improvements in fibrosis. The first step was to identify the most informative markers (including baseline and change from baseline) based on consistent signals across multiple methods including logistic regression, random forests, GUIDE, and regularized regression. A weighted score based on the importance assessed by each method was then formed to rank the markers. In the second step, logistic ridge regression (including baseline and change from baseline as predictors) was used to establish the multi-marker classification algorithm based on the selected markers, and its performance assessed using AUROC. All statistical analyses were performed using R and Graphpad Prism software.

Baseline Associations Between Eicosanoids and Fibrosis Stage

Analysis population: Data from 427 patients with baseline histological data and plasma eicosanoid assessment were used in this analysis. Participants had a mean age of 52 years and BMI of 34 kg/m². The distribution of fibrosis stages was F0: 7%, F1: 22%, F2: 24%, F3: 34%, and F4: 12%. Detailed baseline demographic and clinical characteristics of the participants are provided in Table 1.

TABLE 1
Baseline characteristics of the study population.

| | | | | | NASH with | |
|---|---|---|---|---|---|---|
| | | | NASH with | | advanced | |
| | | | F1 and F2 | | fibrosis | |
| | All | | fibrosis | | (F3-4) | |
| Characteristics | (n = 427) | | (n = 197) | | (n = 197) | |
| Demographics | | | | | | |
| Age, years | 52 | (42-62) | 51 | (40-62) | 55 | (46-64) |
| Male, n (%) | 183 | (43) | 94 | (48) | 69 | (35) |
| White, n (%) | 371 | (87) | 163 | (83) | 180 | (91) |
| Hispanic or | 84 | (20) | 35 | (18) | 41 | (21) |
| Latino, n (%) | | | | | | |
| BMI, kg/m² | 34 | (27-41) | 34 | (28-40) | 35 | (28-42) |
| Clinical | | | | | | |
| Type 2 | 151 | (35) | 35 | (18) | 114 | (58) |
| Diabetes, n (%) | | | | | | |
| Biological data | | | | | | |
| AST (U/L) | 50 | (20-80) | 44 | (18-70) | 58 | (25-91) |
| ALT (U/L) | 64 | (18-110) | 66 | (16-116) | 65 | (24-106) |
| GGT (Ui/L) | 100 | (−32-232) | 68 | (−1-137) | 134 | (−33-301) |
| Albumin (g/dL) | 4 | (4-4) | 4 | (4-4) | 4 | (4-4) |
| Platelet count | 237 | (169-305) | 249 | (185-313) | 224 | (152-296) |
| (10³/μL) | | | | | | |
| Histology | | | | | | |
| Fibrosis n (%) | | | | | | |
| 0 | 32 | (7) | 0 | (0) | 0 | (0) |
| 1 | 96 | (22) | 96 | (49) | 0 | (0) |
| 2 | 101 | (24) | 101 | (51) | 0 | (0) |
| 3 | 146 | (34) | 0 | (0) | 146 | (74) |
| 4 | 51 | (12) | 0 | (0) | 51 | (26) |
| Steatosis n (%) | | | | | | |
| 0 | 42 | (10) | 15 | (8) | 11 | (6) |
| 1 | 262 | (61) | 121 | (61) | 128 | (65) |
| 2 | 105 | (25) | 50 | (25) | 52 | (26) |
| 3 | 17 | (4) | 11 | (6) | 6 | (3) |
| Lobular | | | | | | |
| inflammation | | | | | | |
| n (%) | | | | | | |
| 0 | 1 | (0) | 0 | (0) | 0 | (0) |
| 1 | 74 | (17) | 44 | (22) | 4 | (2) |
| 2 | 192 | (45) | 104 | (53) | 83 | (42) |
| 3 | 159 | (37) | 49 | (25) | 110 | (56) |
| Ballooning n | | | | | | |
| (%) | | | | | | |
| 0 | 108 | (25) | 72 | (37) | 8 | (4) |
| 1 | 141 | (33) | 76 | (39) | 61 | (31) |
| 2 | 177 | (41) | 49 | (25) | 128 | (65) |
| NAS, Median | 5 | (4, 6) | 5 | (4, 6) | 6 | (5, 6) |
| (IQR) | | | | | | |

Median and interquartile range (IQR) values and n (%) are provided unless otherwise noted.
BMI, body mass index; ALT, alanine aminotransferase; AST, aspartate aminotransferase; GGT, Gamma-Glutamyl Transferase; NAS, NAFLD Activity Score.

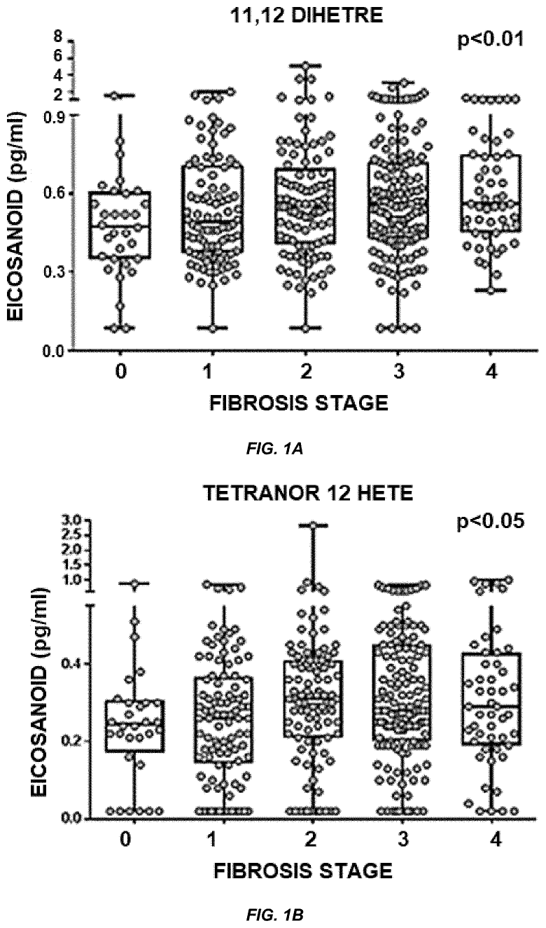

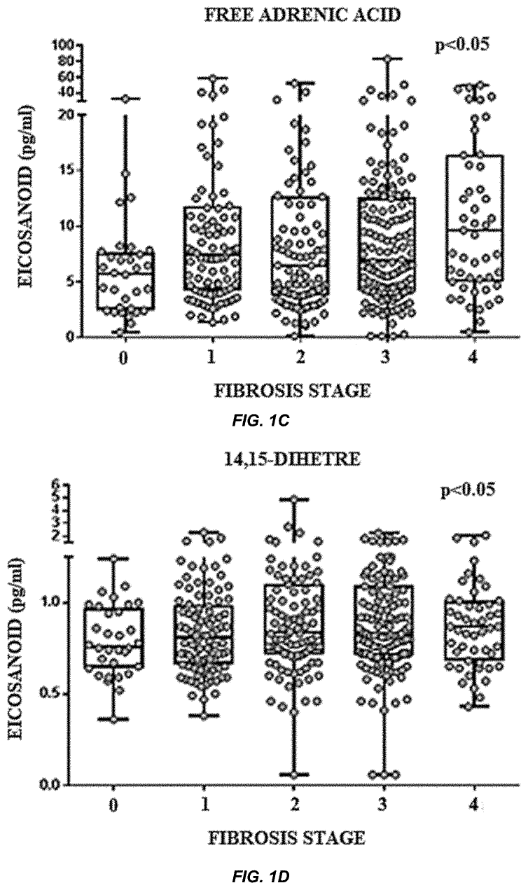

Plasma eicosanoids are associated with fibrosis stage: 4 plasma eicosanoids were identified that were significantly associated with fibrosis stage: 11, 12-DIHETE (p=0.0094), tetranor 12-HETE (p=0.0135), adrenic acid (p=0.0328) and 14, 15-DIHETE (p=0.0481; FIG. 1). In addition, 3 plasma eicosanoids were significantly associated with the presence of advanced fibrosis (stage 3 and 4): 7,17-DHDPA (p=0.01), 11,12-DIHETRE (p=0.03), and DHK-PGD2 (p=0.03; Table 2). Four plasma eicosanoids were significantly associated with cirrhosis (stage 4 fibrosis): 8-HETE (p=0.0103), 11-HETE (p=0.0212), adrenic acid (p=0.0259), and 15-HETRE (p=0.0299; Table 3).

TABLE 2
Eicosanoids that are significantly altered in
NASH subjects with advanced fibrosis (F3/4).

| | Fibrosis stage F0-2 versus F3-4 | | |
|---|---|---|---|
| | F0-2 (n = 229) | F3-4 (n = 196) | |
| Marker | Median (Q1, Q3) | Median (Q1, Q3) | p-value |
| 7,17-DHDPA | 1.06 (0.79, 1.61) | 0.91 (0.68, 1.43) | 0.0114 |
| 11,12- | 0.52 (0.39, 0.67) | 0.56 (0.44, 0.73) | 0.0303 |
| DIHETRE | | | |
| DHK-PGD2 | 12.44 (10.52, 14.77) | 13.16 (11.06, 15.92) | 0.0312 |

TABLE 3
Eicosanoids that are significantly higher
in NASH subjects with cirrhosis (F4).

| | Fibrosis Stage F0-3 versus F4 | | |
|---|---|---|---|
| | F0-3 (n = 374) | F4 (n = 51) | |
| Marker | Median (Q1, Q3) | Median (Q1, Q3) | p-value |
| 8-HETE | 0.53 (0.39, 0.72) | 0.64 (0.5, 0.88) | 0.0077 |
| 11-HETE | 0.68 (0.47, 1.02) | 0.92 (0.61, 1.35) | 0.0152 |
| 15-HETRE | 0.17 (0.13, 0.25) | 0.19 (0.16, 0.3) | 0.0209 |
| Free Adrenic Acid | 7.02 (3.98, 11.96) | 9.63 (4.99, 16.44) | 0.037 |
| 5-HETE | 1.57 (1.12, 2.19) | 1.94 (1.27, 2.48) | 0.043 |

Longitudinal Associations Between Changes of Eicosanoids and Liver Fibrosis Improvement

Analysis population: Of the 72 patients with biopsy-proven NASH and stage 2 or 3 fibrosis who were randomized and treated in the phase 2 trial of selonsertib, 63 with evaluable eicosanoid assessment and liver biopsy at baseline and Week 24 were included in the analysis. Detailed baseline characteristics are provided in Table 4.

TABLE 4
Baseline characteristics of the longitudinal study population.

| | All | |
|---|---|---|
| Characteristics | (n = 63) | |
| Demographics | | |
| Age, years | 54 | (44.64) |
| Male, n (%) | 18 | (29) |
| White, n (%) | 57 | (90) |
| Hispanic or Latino, n (%) | 20 | (32) |
| BMI, kg/m² | 35 | (27-43) |
| Clinical | | |
| Type 2 Diabetes, n (%) | 46 | (73) |
| Biological data | | |
| AST (U/L) | 56 | (32-80) |
| ALT (U/L) | 70 | (38-102) |
| GGT (Ui/L) | 74 | (−30-178) |
| Albumin (g/dL) | 5 | (5-5) |
| Platelet count (10³/μL) | 237 | (176-298) |
| Histology | | |
| Fibrosis n (%) | | |
| 2 | 20 | (32) |
| 3 | 43 | (68) |
| Steatosis grade 2-3 n (%) | 22 | (35) |
| Lobular inflammation grade 3 n (%) | 42 | (67) |
| Ballooning grade 2 n (%) | 55 | (87) |
| NAS 6-8, n (%) | 63 | (100) |
| Hepatic collagen content (%) | 4 | (2-6) |

Median and interquartile range (IQR) values and n (%) are provided, unless otherwise noted.
BMI, body mass index; ALT, alanine aminotransferase; AST, aspartate aminotransferase; GGT, Gamma-Glutamyl Transferase; NAS, NAFLD Activity Score.

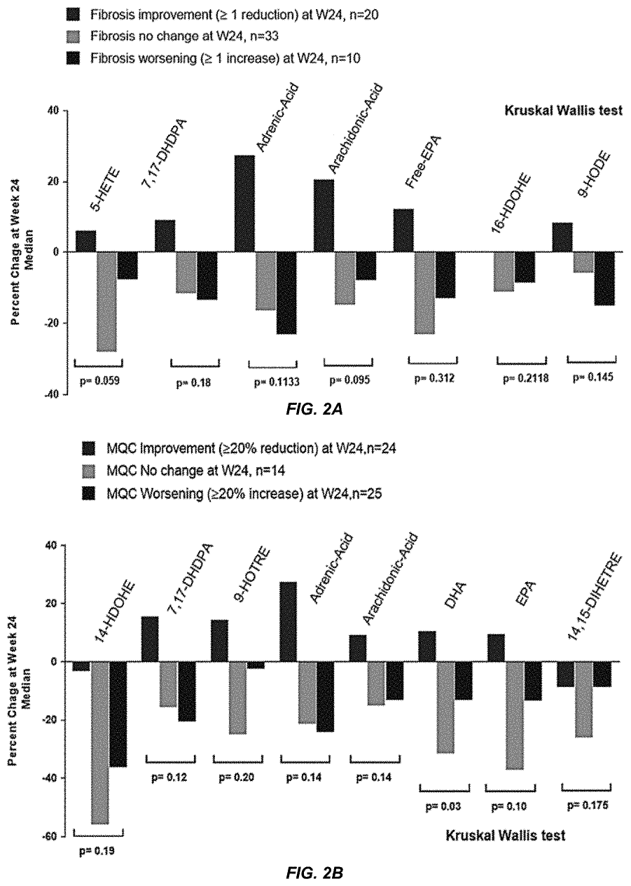

Changes in plasma eicosanoid are associated with liver fibrosis improvement: 7 eicosanoids were identified: 5-HETE, 7, 17-DHDPA, adrenic acid, arachidonic acid, EPA, 16-HDOHE, and 9-HODE as the most informative markers using a weighted score for the prediction of ≥1-stage improvement of fibrosis. The median relative changes of these 7 eicosanoids stratified by liver fibrosis changes (fibrosis improvement, no change and worsening) are shown in FIG. 2A. Baseline and longitudinal change values of these 7 eicosanoids were evaluated as predictors for fibrosis improvement at Week 24. The AUROC of the individual eicosanoids ranged between 0.52 to 0.67 and the combination of the 7 eicosanoids yielded a good diagnostic performance for the prediction of liver fibrosis improvement (AUROC 0.74; 95% CI: 0.62-0.87; Table 5).

TABLE 5
Performance of an eicosanoid panel for the
prediction of liver fibrosis improvement

| | FIBROSIS IMPROVEMENT | | |
|---|---|---|---|
| | (≥1 stage of liver fibrosis reduction) | | |
| Marker | Wilcoxon p | AUC* | 95% CI |
| 5-HETE | 0.0554 | 0.54 | 0.47, 0.63 |
| 7,17-DHDPA | 0.068 | 0.56 | 0.45, 0.62 |
| Adrenic acid | 0.0373 | 0.58 | 0.49, 0.70 |
| Arachidonic acid | 0.0359 | 0.67 | 0.51, 0.70 |
| EPA | 0.131 | 0.60 | 0.49, 0.65 |
| 16-HDOHE | 0.0908 | 0.52 | 0.45, 0.57 |
| 9-HODE | 0.0910 | 0.52 | 0.48, 0.62 |
| 7 Eicosanoids | | 0.74 | 0.62, 0.87 |
| panel | | | |

AUC: Area under the curve derived using baseline value and change from baseline of Eicosanoids as predictors

Changes in plasma eicosanoids are associated with improvement in hepatic collagen content: The associations between changes in plasma eicosanoids and hepatic collagen content were also analyzed. 8 eicosanoids were identified: 14, HDOHE, 7,17-DHDPA, 9,HOTRE, adrenic acid, arachidonic acid, DHA, EPA, and 14, 15-DIHETRE as the most informative markers using a weighted score for the prediction of hepatic collagen content improvement (≥20% relative reduction from baseline to Week 24). The median relative changes of these 7 eicosanoids stratified by hepatic collagen content changes (improvement, no change, and worsening) are shown in FIG. 2B. Baseline and longitudinal changes of these eicosanoids were evaluated as predictors of hepatic collagen content improvement at Week 24. The AUROCs of the top four individual eicosanoids ranged from 0.49 to 0.69 and the combination of these 4 eicosanoids yielded a good diagnostic performance for the prediction of hepatic collagen content improvement with an AUROC of 0.72 (95% CI: 0.50-0.77; Table 6).

TABLE 6
Performance of an eicosanoid panel for the
prediction of hepatic collagen improvement

| | Hepatic collagen improvement (≥20% reduction) | | |
|---|---|---|---|
| Marker | Wilcoxon p | AUC | 95% CI |
| 14-HDOHE | 0.0805 | 0.51 | 0.45, 0.58 |
| 7,17-DHDPA | 0.0431 | 0.63 | 0.58, 0.67 |
| 9-HOTRE | 0.078 | 0.69 | 0.66, 0.73 |
| Adrenic Acid | 0.0462 | 0.49 | 0.44, 0.55 |
| 4 Eicosanoids panel | | 0.72 | 0.50, 0.77 |

AUC: Area under the curve derived from baseline value and change from baseline of Eicosanoids as predictors.

Numerous modifications and variations in the invention as set forth in the above illustrative examples are expected to occur to those skilled in the art. Consequently only such limitations as appear in the appended claims should be placed on the invention.

What is claimed:

1. A method of determining changes in liver fibrosis, comprising:
(a) obtaining a biological sample from the subject;
(b) spiking the sample with deuterated standards;
(c) extracting one or more eicosanoids selected from the group consisting of adrenic acid, 11,12-diHETE, tetranor 12-HETE, 14,15-diHETE and any combination thereof;
(d) measuring the one or more eicosanoids using chromatography and/or gas chromatography mass spectroscopy; and
(e) comparing the levels of adrenic acid, 11,12-diHETE, tetranor 12-HETE and/or 14,15-diHETE in the biological sample obtained from the subject to a control or prior sample, wherein a difference in the levels is indicative of a change in liver fibrosis.

2. The method of claim 1, wherein the biological sample is selected from the group consisting of blood, blood plasma and blood serum.

3. The method of claim 1, further comprising measuring 5-HETE, 7,17-DHDPA, arachidonic acid, EPA, 16-HDOHE and 9-HODE.

4. The method of claim 1, further comprising measuring additional eicosanoid in the sample obtained from the subject.

5. The method of claim 1, wherein the one or more eicosanoids are measured by liquid chromatography.

6. The method of claim 1, wherein the one or more eicosanoids are measured by gas chromatography mass spectrometry.

7. The method of claim 1, further comprising determining the area under receiver operating characteristic curve (AUROC) based upon a ratio of the levels of the one or more eicosanoids matched with the deuterated standards of the same eicosanoid.

8. A method of determining liver fibrosis improvement, comprising:
(a) obtaining a biological sample from the subject;
(b) spiking the sample with deuterated standards;
(c) extracting one or more eicosanoids selected from the group consisting of 5-HETE, 7,17-DHDPA, adrenic acid, arachidonic acid, EPA, 16-HDOHE, 9-HODE and any combination thereof;
(d) measuring the one or more eicosanoids using chromatography and/or gas chromatography mass spectroscopy; and
(e) comparing the levels of 5-HETE, 7,17-DHDPA, adrenic acid, arachidonic acid, EPA, 16-HDOHE, and/or 9-HODE in the biological sample obtained from the subject to a control or prior sample, wherein a positive percent change in the levels is indicative of an improvement in liver fibrosis.

9. The method of claim 7, wherein the biological sample is selected from the group consisting of blood, blood plasma and blood serum.

10. The method of claim 7, further comprising measuring one or more of 11,12-diHETE, tetranor 12-HETE, 14,15-diHETE, 14-HDOHE, 9-HOTRE, DHA, and/or EPA.

11. The method of claim 7, wherein the one or more eicosanoids are measured by liquid chromatography.

12. The method of claim 7, wherein the one or more eicosanoids are measured by gas chromatography mass spectrometry.

13. The method of claim 7, further comprising determining the area under receiver operating characteristic curve (AUROC) based upon a ratio of the levels of the one or more eicosanoids matched with the deuterated standards of the same eicosanoid.

14. A method of determining an improvement in hepatic collagen content, comprising:
(a) obtaining a biological sample from the subject;
(b) spiking the sample with deuterated standards;
(c) extracting one or more eicosanoids selected from the group consisting of 14-HDOHE; 7,17-DHDPA; 9HOTRE; adrenic acid; arachidonic acid; DHA; EPA; 14,15-DiHETRE and any combination thereof;
(d) measuring the one or more eicosanoids using chromatography and/or gas chromatography mass spectroscopy; and
(e) comparing the levels of 14-HDOHE; 7,17-DHDPA; 9HOTRE; adrenic acid; arachidonic acid; DHA; EPA; and/or 14,15-DiHETRE in the biological sample obtained from the subject to a control or prior sample, wherein a positive percent change in the levels is indicative of an improvement in liver fibrosis.

15. The method of claim 14, wherein the biological sample is selected from the group consisting of blood, blood plasma and blood serum.

16. The method of claim 14, further comprising measuring one or more of 5-HETE, 16-HDOHE, and/or 9-HODE.

17. The method of claim 14, wherein the one or more eicosanoids are measured by liquid chromatography.

18. The method of claim 14, wherein the one or more eicosanoids are measured by gas chromatography mass spectrometry.

19. The method of claim 14, further comprising determining the area under receiver operating characteristic curve (AUROC) based upon a ratio of the levels of the one or more eicosanoids matched with the deuterated standards of the same eicosanoid.

20. A method of determining improved prognosis of liver fibrosis comprising measuring eicosanoids selected from the group consisting of 5-HETE, 7,17-DHDPA, adrenic acid, arachidonic acid, EPA, 16-HDOHE, 9-HODE in a sample from a subject at a first time point;
treating the subject with a therapeutic for the treatment of fibrotic liver disease;
measuring eicosanoids selected from the group consisting of 5-HETE, 7,17-DHDPA, adrenic acid, arachidonic acid, EPA, 16-HDOHE, 9-HODE in a sample from a subject at a second time point after treating the subject;
wherein if there is a positive percent increase in 5-HETE, 7,17-DHDPA, adrenic acid, arachidonic acid, EPA, 16-HDOHE, and/or 9-HODE, the therapeutic is treating liver fibrosis in the subject.
